(19) United States
(12) Patent Application Publication
(10) Pub. No.: US 2011/0244023 A1
(43) Pub. Date: Oct. 6, 2011

(54) TRANSDERMAL DELIVERY PATCH

(75) Inventors: Jeremy Cottrell, Caulfield South (AU); Giacinto Gaetano, South Melbourne (AU); Mahmoud El-Tamimy, Meadow Heights (AU); Nicholas Kennedy, Boronia (AU); Paul David Gavin, Chadstone (AU)

(73) Assignee: PHOSPHAGENICS LIMITED, Melbourne (AU)

(21) Appl. No.: 12/782,564

(22) Filed: May 18, 2010

Related U.S. Application Data
(60) Provisional application No. 61/319,007, filed on Mar. 30, 2010.

(51) Int. Cl. A61K 9/70; A61K 38/28; A61K 47/30; A61K 47/34; A61K 47/32; A61P 43/00

(52) U.S. Cl. 424/449; 514/772; 514/772.3; 514/772.4; 514/772.7; 514/6.5

(57) ABSTRACT

A composition suitable for use in a transdermal delivery patch for administration of a biologically active compound, the composition comprising a phosphate compound of tocopherol and a polymer carrier.

### CROSS-REFERENCE TO RELATED APPLICATIONS

This application claims the benefit of U.S. Provisional Application No. 61/319,007 filed Mar. 30, 2010, the content of which is incorporated herein by reference in its entirety.

### TECHNICAL FIELD

The present invention relates to a transdermal delivery patch for administration of biologically active compounds. More specifically, the present invention relates to a transdermal delivery matrix patch for administration of these compounds.

### BACKGROUND

In this specification where a document, act or item of knowledge is referred to or discussed, this reference or discussion is not an admission that the document, act or item of knowledge or any combination thereof was at the priority date, publicly available, known to the public, part of common general knowledge; or known to be relevant to an attempt to solve any problem with which this specification is concerned.

Drug delivery is the method or process of administering a pharmaceutical compound to achieve a therapeutic effect in humans and animals.

Drug delivery technologies have been developed to improve bioavailability, safety, duration, onset or release, of the pharmaceutical compound.

When developing drug delivery technologies, problems likely to be encountered include compatibility of the drug delivery system and the pharmaceutical compound, maintaining an adequate and effective duration, potential for side effects, and meeting patient convenience and compliance. As a consequence, many drug delivery technologies fall short of desired improvements and requirements.

Accordingly, there is still a need for alternate drug delivery systems that effectively deliver drugs.

### SUMMARY

It has surprisingly been found that biologically active compounds can be effectively administered using a transdermal delivery matrix patch.

According to a first aspect, there is provided a composition suitable for use in a transdermal delivery patch for administration of a biologically active compound, the composition comprising a phosphate compound of tocopherol and a polymer carrier.

In one embodiment, the transdermal delivery patch is a matrix patch.

A second aspect provides use of a phosphate compound of tocopherol and a polymer carrier as a matrix layer in a transdermal delivery patch for administration of a biologically active compound.

The phosphate compound of tocopherol may be selected from the group consisting of mono-(tocopheryl)phosphate, mono-(tocopheryl)phosphate monosodium salt, mono-(tocopheryl)phosphate disodium salt, mono-(tocopheryl)phosphate monopotassium salt, mono-(tocopheryl)phosphate dipotassium salt, di-(tocopheryl)phosphate, di-(tocopheryl)phosphate monosodium salt, di-(tocopheryl)phosphate monopotassium salt, or a mixture thereof. These phosphate compounds may be derived from the alpha, beta, gamma or delta form of tocopherol, or a combination thereof.

The composition, or matrix layer, may comprise a phosphate compound of tocopherol in an amount within the range of about 0.01% w/w to about 10% w/w, about 0.1% w/w to about 5% w/w, about 0.5% w/w to about 2% w/w or to about 3% w/w, of the total concentration of the matrix layer. In one embodiment, the phosphate compound of tocopherol is present in an amount of about 1% w/w to about 1.5% w/w of the total concentration of the composition, or matrix layer.

The polymer carrier may comprise natural and synthetic polymers, co-polymers, or terpolymers. Preferred polymer carriers that are suitable for use in the composition, or matrix layer, include polyvinyl pyrrolidone (e.g. PVP K90, MW 360,000 Da), polysiloxanes and polymethyl methacrylate (e.g. Eudragit E100). The composition, or matrix layer, may comprise a polymer carrier in an amount of from about 20% w/w up to about 90% w/w, from about 30% w/w up to about 80% w/w, from about 55% w/w up to about 65% w/w, of the total weight of the composition, or matrix layer.

The polymer carrier may also comprise inert carrier components selected from the group consisting of anti-tacking agents, tackifiers, and plasticizers.

Inert carrier components may be present in the composition, or matrix layer, in an amount of from 0.001% w/w up to about 50% w/w, up to about 40% w/w, up to about 30% w/w, of the total weight of the composition, or matrix layer.

A third aspect provides a transdermal delivery matrix patch for administration of a biologically active compound comprising (i) a backing layer, and (ii) a matrix layer, which comprises a phosphate compound of tocopherol and a polymer carrier (as defined above), and a biologically active compound.

A fourth aspect provides use of a matrix patch for transdermal delivery of a biologically active compound, the matrix patch comprising (i) a backing layer and (ii) a matrix layer, which comprises a phosphate compound of tocopherol and a polymer carrier (as defined above), and a biologically active compound.

The biologically active compound may be selected from the group consisting of pharmaceuticals including drugs, cosmeceuticals, nutraceuticals, and nutritional agents.

Preferably the backing layer is occlusive.

A fifth aspect provides a method for preparing a transdermal delivery matrix patch for administration of a biologically active compound comprising the steps of:
(i) combining a polymer carrier and optional inert carrier components with a suitable solvent;
(ii) combining (i) with a dispersion comprising a phosphate compound of tocopherol and a biologically active compound;
(iii) stirring (ii) until complete homogenisation is achieved;
(iv) placing (iii) in a mould comprising a suitable backing layer; and
(v) drying the compositions in the mould by heating them up to about 90° C. for about 0.5 to about 24 hours.

Preferably, the drying is conducted at a temperature of 75° C.

### BRIEF DESCRIPTION OF THE DRAWINGS

The examples will be described with reference to the accompanying figures in which:

FIG. 1 is a schematic diagram of a matrix patch of one embodiment of the present invention;

FIG. 2 is a graph showing the average change in blood glucose after application of matrix patches of the present invention to each of the animals;

FIG. 3 is a graph showing the area under the curve of the graph of FIG. 2;

FIGS. 4 and 5 are graphs comparing the deposition in skin of two diclofenac matrix patches; and

FIG. 6 is a graph comparing the permeation of two lidocaine matrix patches.

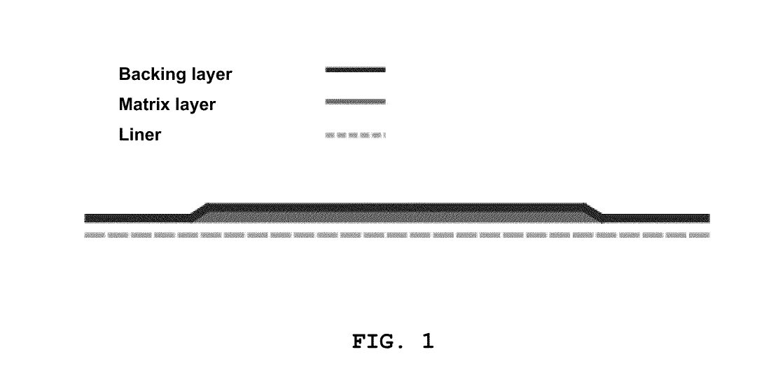

### DETAILED DESCRIPTION

The present invention relates to a composition suitable for use in a transdermal delivery matrix patch for administration of a biologically active compound, the composition comprising a phosphate compound of tocopherol and a polymer carrier. The composition, or matrix layer, may form part of a transdermal delivery matrix patch. It has been surprisingly found that a transdermal delivery matrix patch comprising this matrix layer can effectively administer biologically active compounds.

### Phosphate Compound of Tocopherol

The composition, or matrix layer, comprises a phosphate compound of tocopherol.

Vitamin E exists in eight different forms, namely four tocopherols and four tocotrienols. All feature a chroman ring, with a hydroxyl group that can donate a hydrogen atom to reduce free radicals and a hydrophobic side chain which allows for penetration into biological membranes. Such derivatives of vitamin E may be classified as “hydroxy chromans”. Both tocopherols and tocotrienols occur in alpha, beta, gamma and delta forms, determined by the number and location of methyl groups on the chroman ring. The tocotrienols differ from the analogous tocopherols by the presence of three double bonds in the hydrophobic side chain. The various forms of vitamin E are shown by Formula (I):

(I)

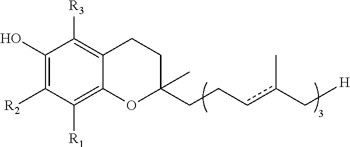

| | R₁ | R₂ | R₃ |
|---|---|---|---|
| α-tocopherol | CH₃ | CH₃ | CH₃ |
| α-tocotrienol | | | |
| β-tocopherol | CH₃ | H | CH₃ |
| β-tocotrienol | | | |
| γ-tocopherol | H | CH₃ | CH₃ |
| γ-tocotrienol | | | |
| δ-tocopherol | H | H | CH₃ |
| δ-tocotrienol | | | |

In the present invention, tocopherol in any of the four forms may be used. The alpha form of tocopherol is preferred.

The term “phosphate compound” refers to phosphorylated tocopherol, where a covalent bond is formed between an oxygen atom (typically originating from a hydroxyl group) of the tocopherol compound and the phosphorous atom of a phosphate group (PO₄). The phosphate compound may be a phosphate mono-ester, phosphate di-ester, phosphate tri-ester, pyrophosphate mono-ester, pyrophosphate di-ester, or a salt or derivative thereof, or a mixture thereof. The di- and tri-esters may comprise the same tocopherol form or different tocopherol forms.

The “salts” include metal salts such as alkali or alkaline earth metal salts, for example sodium, magnesium, potassium and calcium salts. Sodium and potassium salts are preferred.

The “derivatives” include phosphate compounds where one or more phosphate protons are replaced by a substituent. Some non-limiting examples of derivatives include phosphatidyl derivatives where a phosphate proton is substituted with an amino-alkyl group, sugar derivatives where a phosphate proton is substituted with a sugar such as glucose.

The term “amino-alkyl group” refers to a group comprising an amino (—NH₂) group and an alkyl group. The term “alkyl” refers to straight chain, branched chain or cyclic hydrocarbon groups having from 1 to 8 carbon atoms. Examples include methyl, ethyl, propyl, isopropyl, butyl, isobutyl, sec-butyl, tert-butyl, pentyl, hexyl, cyclohexyl, heptyl, and octyl. Phosphatidyl choline derivatives are most preferred.

The phosphate compounds of tocopherol may be selected from the group consisting of mono-(tocopheryl)phosphate, mono-(tocopheryl)phosphate monosodium salt, mono-(tocopheryl)phosphate disodium salt, mono-(tocopheryl)phosphate monopotassium salt, mono-(tocopheryl)phosphate dipotassium salt, di-(tocopheryl)phosphate, di-(tocopheryl)phosphate monosodium salt, di-(tocopheryl)phosphate monopotassium salt, or a mixture thereof. These phosphate compounds may be derived from the alpha, beta, gamma or delta form of tocopherol, or a combination thereof.

When a mixture of a mono-phosphate ester and a di-phosphate ester, that is a mono-(tocopheryl)phosphate and di-(tocopheryl) phosphate (which may in some instances herein be simply referred to as “TPM”), the ratio is preferably at least 2:1, more preferably within the range of about 4:1 to about 1:4, most preferably within the range of about 6:4 to about 8:2. The ratio may be about 6:4 or about 8:2.

The matrix layer may comprise a phosphate compound of tocopherol in an amount within the range of about 0.01% w/w to about 10% w/w, about 0.1% w/w to about 5% w/w, about 0.5% w/w to about 2% w/w or to about 3% w/w, of the total concentration of the matrix layer. In one embodiment, the phosphate compound of tocopherol is present in an amount of about 1% w/w to about 2% w/w of the total concentration of the matrix layer.

### Polymer Carrier

The composition, or matrix layer, also comprises a polymer carrier.

The polymer carrier may comprise natural and synthetic polymers, co-polymers, or terpolymers.

Natural polymers include rubbers, elastomers, polysaccharides such as cellulose, natural resins such as shellac and amber.

Synthetic polymers include, for example, polyacrylates, polyamides, polyesters, polycarbonates, polyimides, polystyrenes, acrylonitrile butadiene styrene, polyacrylonitrile, polybutadiene, poly(butylene terephthalate), poly(ether sulphone), poly(ether)ketones, polyethylene, poly(ethylene glycol), poly(ethylene terphthalate), polypropylene, polytetratfluoroethylene, styrene-acrylonitrile resin, poly(trimethylene terephthalate), polyurethanes, polyvinyl butyral, polyvinylchlorides, polyvinylidenedifluoride, poly(vinyl pyrrolidone), polychloroprene, fluoroelastomers, chloro-sulphonated rubbers, hypromellose, polyolefine elastomer, polyacrylamide, chlorinated polyethylene, polyethersulphone, nylon, liquid crystal polymers, polyethylene terephthalate (PET), polyphenylsulphone, polypthalaminepolyvinyl alcohol derivatives, poly ethylene glycols, ethylene vinyl acetate, polymethyl methacrylate, cellulose derivatives such as ethyl cellulose, hydroxylpropyl methyl cellulose, sugar derivatives (gums) including derivatives of sorbitol and mannitol, and silicone oil derivatives such as polysiloxanes.

Preferred polymer carriers that are suitable for use in the matrix layer of the present invention include polyvinyl pyrrolidone (e.g. PVP K90, MW 360,000 Da), polysiloxanes and polymethyl methacrylate (e.g. Eudragit E100). In one embodiment, the polymer carrier is polyvinyl pyrrolidone. In an alternate embodiment, the polymer carrier is polymethyl methacrylate.

The polymer carrier used in the matrix layer may have sufficient tackiness to enable the matrix patch to adhere to skin. For instance, amine-resistant polysiloxanes and mixtures thereof can be used in the matrix layer. A mixture of a polysiloxane of medium tack and a polysiloxane of high tack is used would be most suitable. The polysiloxanes may be synthesized from linear bifunctional and branched polyfunctional oligomers. It has been found that the ratio of both types of oligomers determines the physical properties of the polymers. More polyfunctional oligomers result in a more cross-linked polymer with a higher cohesion and a reduced tack, less polyfunctional oligomers result in a higher tack and a reduced cohesion. A high tack version should be tacky enough for the matrix patch to adhere to the surface of skin. A medium tack version, on the other hand, may not be tacky at all but could be useful by providing a softening effect to other components included in the matrix layer. To increase the adhesive power of the matrix layer, a silicone oil (e.g. dimethicone) could be added.

The matrix layer may comprise a polymer carrier in an amount of from about 20% w/w up to about 95% w/w, from about 30% w/w up to about 80% w/w, from about 55% w/w up to about 65% w/w, of the total weight of the matrix layer. In one specific embodiment, the matrix layer may comprise a polymer carrier in an amount of from about 85% w/w up to about 95% w/w, of the total weight of the matrix layer.

The polymer carrier may also comprise inert carrier components, such as for example, anti-tacking agents, tackifiers, and plasticizers to achieve appropriate softness, flexibility and “tackiness” for the polymer carrier to enable the matrix layer to adhere to the surface of skin, and thus provide consistent delivery.

For polymers which are naturally “tacky” and may need anti-tackiness to have an appropriate consistency, anti-tacking agents that are solid with no stickiness property (i.e. low ability to retain solvents upon drying) and that can be mixed well (i.e. do not crystallise upon drying) with the polymer carrier may be suitable. The selection would be based on the polymer-type. Many surfactants are suitable for use as an anti-tacking agent with a polymer carrier. A more specific example of an anti-tacking agent is succinic acid.

In order to enhance the ability of the matrix layer to adhere to the surface of skin, it may optionally contain a tackifier (or tacking agent). Tack can be controlled by combining adhesives of varying hardnesses (glass temperature or T_{g}). Typically, a tackifier is a polymer which is insoluble in water and composed of a monomer which contains partly or wholly a (meth)acrylic alkyl ester. Such types of polymers include, but are not limited to, acrylic, N-butyl-methacrylic copolymer (Primal N580NF, sold by Japan Acrylic Chemical Company, Ltd.), acrylic methyl, acrylic 2-ethylhexyl copolymer (Nikasol TS-6520, sold by Nippon Carbide Industries Company, Ltd), polyacrylic acid (Jurymer AC-IOLPH, sold by Nihon Junyaku Company, Ltd), methacrylic copolymer L (Plastoid L50, sold by Rohm Pharma GmbH), and aminoalkylmethacrylate copolymer E (Plastoid E35L, Plastoid E35M, Plastoid E35H, all sold by Rohm Pharma GmbH). Other non-limiting examples include rosin esters, hydrogenated rosins, dipropylene glycol dibenzoate, and/or mixed hydrocarbons, and acrylic copolymers (e.g. Flexbond 150 adhesive by Air Products).

Plasticizers are additives that increase the plasticity or fluidity of the material to which they are added. Plasticizers may be used in the present invention to soften the final product increasing its flexibility and making it less brittle. Suitable plasticizers include phthalates, esters of polycarboxylic acids with linear or branched aliphatic alcohols of moderate chain length, acetylated monoglycerides, alkyl citrates, triethyl citrate (TEC), acetyl triethyl citrate (ATEC), tributyl citrate (TBC), acetyl tributyl citrate (ATBC), trioctyl citrate (TOC), acetyl trioctyl citrate (ATOC), trihexyl citrate (THC), acetyl trihexyl citrate (ATHC), butyryl trihexyl citrate (BTHC, trihexyl o-butyryl citrate), trimethyl citrate (TMC), alkyl sulphonic acid phenyl ester, bis(2-ethylhexyl) phthalate (DEHP), bis(n-butyl)phthalate (DnBP, DBP), diisooctyl phthalate (DIOP), bis(n-butyl)phthalate (DnBP, DBP), diisobutyl phthalate (DIBP), bis(2-ethylhexyl)adipate (DEHA), dimethyl adipate (DMAD), monomethyl adipate (MMAD), dioctyl adipate (DOA), dibutyl sebacate (DBS), dibutyl maleate (DBM), diisobutyl maleate (DIBM), benzoates, epoxidized vegetable oils, N-ethyl toluene sulfonamide (o/p ETSA), N-(2-hydroxypropyl)benzene sulfonamide (HP BSA), N-(n-butyl) benzene sulfonamide (BBSA-NBBS), tricresyl phosphate (TCP), tributyl phosphate (TBP), triethylene glycol dihexanoate (3G6, 3 GH), tetraethylene glycol diheptanoate (4G7), and polyvinylpyrrolidone. Dibutyl sebacate (DBS) is a preferred plasticizer.

Inert carrier components may be present in the matrix layer in an amount of from 0.001% w/w up to about 50% w/w, up to about 40% w/w, up to about 30% w/w, of the total weight of the matrix layer. In one embodiment, the matrix layer comprises an anti-tacking agent (such as succinic acid) and a plasticizer (such as dibutyl sebacate) in a total amount of about 35% w/w of the total weight of the matrix layer.

The amount of polymer carrier and optional inert carrier components present in the matrix layer will depend on the specific biologically active compound to be administered. Generally, however, the matrix layer may comprise these components in an amount of from about 50% w/w up to about 99% w/w, from about 80% w/w up to about 98% w/w, from about 90% w/w up to about 98% w/w, of the total weight of the matrix layer. In one embodiment, the matrix layer comprises these components in the amount of about 95% w/w of the total weight of the matrix layer.

It should be noted that, in some instances herein, the term “polymer carrier” could be used collectively to refer to the polymer carrier and the inert carrier components.

### Additional Optional Components

The matrix layer may optionally further comprise one or more excipients (in addition to the inert carrier components discussed above).

A person skilled in the art of the invention would appreciate what are suitable excipients for inclusion in the matrix layer of the invention. Some examples include, but are not limited to, solvents, thickeners or gelling agents, preservatives, surfactants, stabilizers, plasticizers, adhesives or glues, buffers, emollients, colours, fragrances, and appearance modifiers. It will be appreciated that any excipients which have been approved for use in pharmaceutical products by the regulatory bodies may be employed in the matrix layers (or compositions) of the present invention. The amount of a particular excipient or excipients to be used in a matrix layer of the present invention would also be appreciated by a person skilled in the art.

### Biologically Active Compounds

It has been surprisingly found that the transdermal delivery matrix patch of the present invention can effectively administer biologically active compounds.

The term “biologically active compound” refers to any chemical substance that has a biological effect in humans or animals for medical, therapeutic, cosmetic and veterinary purposes, and encompasses pharmaceuticals including drugs, cosmeceuticals, nutraceuticals, and nutritional agents. It will be appreciated that some of biologically active compounds can be classified in more than one of these classes.

A wide range of biologically active compounds may be delivered with a transdermal delivery matrix patch of the present invention. Examples include, but are not limited to, cardiovascular drugs, in particular antihypertensive agents (e.g. calcium channel blockers (or calcium antagonists)) and antiarrhythmic agents; antibacterial agents; antiinflammatory agents, in particular non-steroidal antiinflammatory agents (NSAIDs), more particularly COX-2 inhibitors, and steroidal antiinflammatory agents; pain management agents, in particular anaesthetics and analgesics; antihyperlipidemic agents, in particular statins; and drugs that affect the central nervous system (CNS) such as antipsychotic drugs such as clozapine, antiepileptic, antianxiety and antiseizure drugs (anticonvulsants), psychoactive drugs, stimulants, antidepressant drugs; hormones such as insulin; vaccines; nutritional agents including vitamins; anticancer drugs; antiviral drugs; anti-Alzheimer drugs; and cosmeceutical drugs such as antibacterial and anti-acne drugs.

A person skilled in the art of the invention would be able to determine whether or not a particular biologically active compound would be suitable for use with a transdermal delivery matrix patch of the present invention. Some specific non-limiting examples of suitable biologically active compounds include:

### Anaesthetics:

amino-ester anaesthetics such as benzocaine, chloroprocaine, cocaine, cyclomethycaine, dimethocaine/larocaine, propoxycaine, procaine/novocaine, proparacaine and tetracaine/amethocaine; amino-amides such as anaesthetics including articaine, bupivacaine, carticaine, cinchocaine/dibucaine, etidocaine, levobupivacaine, lidocaine/lignocaine, mepivacaine, piperocaine, prilocalne, ropivacaine and trimecaine,

### Calcium Channel Blockers:

dihydropyridine calcium channel blockers such as amlodipine, aranidipine, azelnidipine, barnidipine, benidipine, cilnidipine, clevidipine, cronidipine, darodipine, dexniguldipine, efonidipine, elnadipine, elgodipine, felodipine, flordipine, furnidipine, iganidipine, isradipine, lacidipine, lemildipine, lercanidipine, manidipine, mesuldipine, nicardipine, nifedipine, niguldipine, niludipine, nilvadipine, nimodipine, nisoldipine, nitrendipine, olradipine, oxodipine, palonidipine, pranidipine, sagandipine, sornidipine, teludipine, tiamdipine, trombodipine and watanidipine; phenylalkylamine calcium channel blockers such as verapamil and gallopamil, benzothiazepine calcium channel blockers such as diltiazem; and nonselective calcium channel blockers such as mibefradil, bepridil, fluspirilene and fendiline,

### Antiarrhythmic Agents:

amiodarone, disopyramide, flecamide acetate and quinidine sulphate,

### Antibacterial, Antibiotic and Anti Acne Agents:

amoxicillin, azithromycin, benethamine penicillin, benzoyl peroxide, cinoxacin, ciprofloxacin, clarithromycin, clindamycine, clofazimine, chlorohexidine gluconate, cloxacillin, demeclocycline, doxycycline, erythromycin, ethionamide, imipenem, indomethacin, lymocycline, minocycline, nalidixic acid, nitrofurantoin, penicillin, rifampicin, spiramycin, sodium sulfacetamide, sulphabenzamide, sulphadoxine, sulphamerazine, sulphacetamide, sulphadiazine, sulphafurazole, sulphamethoxazole, sulphapyridine, tetracycline, cephalexin, cefdinir, triclosan, ofloxacin, vancocin, glyburide, mupirocin, cefprozil, cefuroxime axetil and trimethoprim,

### Anticancer:

doxorubicin, paclitaxel, docetaxel, camptothecin, megestrol acetate, fluorouracil, teniposide and etopside,

### Antidepressants, Antipsychotics and Antianxiety:

alprazolam, amoxapine, bentazepam, bromazepam, clorazipine, clobazam, clotiazepam, diazepam, lorazepam, flunitrazepam, flurazepam, lormetazepam, medazepam, nitrazepam, oxazepam, temazepam, maprotiline, mianserin, nortriptyline, risperidone, sertraline, trazodone, baloperidol, trimipramine maleate fluoxetine, ondansetron, midazolam, chlorpromazine, triazolam, clozapine, fluopromazine, fluphenazine decanoate, fluanisone, perphenazine, pimozide, prochlorperazine, sulpiride, thioridazine, paroxitine, citalopram, bupropion, divalproex sodium and venlafaxine,

### Tricyclics:

azothiopine, amtriptyline, famotidine, promethazine, paroxatine, oxcarbazapine and mertazapine,

### Antidiabetics:

acetohexamide, chlorpropamide, glibenclaraide, gliclazide, glipizide, metformin, tolazamide, glyburide, glimepiride and tolbutamide,

### Antiepileptics:

beclamide, carbamazepine, clonazepam, ethotoin, methoin, methsuximide, methylphenobarbitone, oxcarbazepine, paramethadione, phenacemide, phenobarbitone, phenyloin, phensuximide, primidone, sulthiamine, phenyloin sodium, nirofurantoin monohydrate, lamotrigine and valproic acid,

### Hypnotics/Sedatives and Muscle Relaxants:

zolpidem tartrate, amylobarbitone, barbitone, butobarbitone, pentobarbitone, brotizolam, carbromal, chlordiazepoxide, chlormethiazole, ethinamate, meprobamate, methaqualome, cyclobenzaprene, cyclobenzaprine, tizanidine and butalbital,

### Antifungal Agents and Antiprotazoal Agents:

amphotericin, butoconazole nitrate, clotrimazole, econazole nitrate, fluconazole, flucytosine, griseofulvin, itraconazole, ketoconazole, miconazole, natamycin, nystatin, sulconazole nitrate, terbinafine, terconazole, tioconazole and undecenoic acid; benznidazole, clioquinol, decoquinate, diiodohydroxyquinoline, diloxanide furoate, dinitolmide, furzolidone, metronidazole, nimorazole, nitrofurazone, ornidazole, terbinafine, clotrimazole, ketoconazole and timidazole,

### Antihypertensive Agents:

candesartan, hydralazine, clonidine, triamterene, felodipine, gemfibrozil, fenofibrate, doxazosin and cilexetil,

### Antimigraine Agents:

dihydroergotamine mesylate, ergotamine tartrate, methysergide maleate, pizotifen maleate and sumatriptan succinate,

### Antimuscarinic Agents:

atropine, benzhexyl, biperiden, ethopropazine, hyoscyamine, mepenzolate bromide, oxyphencylcimine and tropicamide,

### Antineoplastic Agents (or Immunosuppressants):

aminoglutethimide, amsacrine, azathioprine, busulphan, chlorambucil, cyclosporin, dacarbazine, estramustine, etoposide, lomustine, melphalan, mercaptopurine, methotrexate, mitomycin, mitotane, mitozantrone, procarbazine, tamoxifen citrate, testolactone, tacrolimus, mercaptopurine and sirolimus,

### Antiparkinsonian Agents:

bromocriptine mesylate, carbidopa and lysuride maleate,

### Antithyroid Agents:

carbimazole and propylthiouracil,

### Antiviral Drugs:

retinovir, acyclovir, famciclovir, ribavirin, amprenavir, indinavir and efavirenz,

Cardiac Inotropic Agents: aminone, digitoxin, digoxin, enoximone, lanatoside C and medigoxin,

### Hypolipidemic Agents:

fenofibrate,

### Antiinflammatory:

Meoxicam and meloxicam,

### Antihistamine:

fexofenadine, hydroxyzine, promethazine, cetirazine and promethazine,

### Antiulcer:

omeprazole, pantoprazole and ranitidine,

### Diuretics:

Hydrochlorothiazide, furosemide and torsemide,

### NSAIDs:

arylalkanoic acid sub-group of class which includes diclofenac, aceclofenac, acemetacin, alclofenac, bromfenac, etodolac, indometacin, indometacin farnesil, nabumetone, oxametacin, proglumetacin, sulindac and tolmetin; 2-arylpropionic acid (profens) sub-group of class which includes alminoprofen, benoxaprofen, carprofen, dexibuprofen, dexketoprofen, fenbufen, fenoprofen, flunoxaprofen, flurbiprofen, ibuprofen, ibuproxam, indoprofen, ketoprofen, ketorolac, loxoprofen, miroprofen, naproxen, oxaprozin, pirprofen, suprofen, tarenflurbil and tiaprofenic acid; and N-arylanthranilic acid (fenamic acid) sub-group of class which includes flufenamic acid, meclofenamic acid, mefenamic acid and tolfenamic acid; celecoxib, tramadol and propoxyphene,

### Retinoids:

first generation retinoids such as retinol, retinal, tretinoin (retinoic acid, Retin-A), isotretinoin and alitretinoin; second generation retinoids such as etretinate and its metabolite acitretin; third generation retinoids such as tazarotene, bexarotene and adapalene,

### Hormones and Steroids:

adrenocorticotrophic hormone (ACTH), antidiruetic hormone (vasopressin), atrial-nartreuretic peptide (ANP), atrial-nartreuretic factor (ANF), catecholamines, cholecystokinin, clomiphene citrate, danazol, dexamethasone, diethylstilbestrol (DES), ethinyl estradiol, fludrocortison, finasteride, follicle stimulating hormone, gastrin, growth hormone, insulin, leptin, luteinizing hormone, medroxyprogesterone acetate, mestranol, methyltestosterone, nandrolone, norethindrone, norethisterone, norgestrel, estradiol, conjugated oestrogens, oxandrolone, oxytocin, prednisone, progesterone, prolactin, protoglandins, somatostatin, stanozolol, stibestrol, thyroxine, prednisolone phosphate, triamcinolone, acetonide, budesonide, levothyroxine, testosterone and tibolone,

### Statins:

atorvastatin, fluvastatin, lovastatin, nystatin, rosuvastatin, pravastatin and simvastatin,

### Stimulants:

amphetamine, dexamphetamine, dexfenfluramine, fenfluramine and mazindol,

### Vasocontrictors:

desmopressin,

### Vasodilitors:

carvedilol,

### Anti-Alzheimers:

levetiracetam, levitiracetam and donepezil,

### ACE Inhibitors:

benzapril, enalapril, fosinopril sodium, lisinopril, rampril and quinapril,

### Beta Adrenoreceptor Antogonists:

atenolol, propanolol hydrochloride, bisoprolol, metoprolol succinate, metoprolol, and metoprolol tartrate,

### Alcohols and Phenols:

tramadol, tramadol hydrochloride, allopurinol, calcitriol, cilostazol, soltalol, urasodiol bromperidol, droperidol, flupenthixol decanoate, albuterol, albuterol sulphate, carisoprodol, chlobetasol, ropinirol, labetalol, and methocarbamol,

### Ketones:

amioderone, fluticasone, spironolactone, prednisone, triazodone, desoximetasone, methyl prednisdone, nabumetone and buspirone,

### Cosmetic Peptides:

acetyl hexapeptide-3, hyaluronic acid, acetyl octapeptide and 1-carnosine,

### Vaccines:

vaccines comprising toxoids (inactivated toxic compounds); proteins, protein subunits and polypeptides; polynucleotides such as DNA and RNA; conjugates; adjuvants such as saponins, virosomes, inorganic and organic adjuvants, for example zostavax,

### Nutraceutical Actives:

coenzyme Q₁₀ (or ubiquinone), ubiquinol or resveratrol; a carotenoid, which may be selected from α-, β-, or γ-carotene, lycopene, lutein, zeaxanthin and astaxanthin; a phytonutrient, which may be selected from lycopene, lutein and seaxanthin; an unsaturated fatty acid, which may be linoleic acid, conjugated linoleic acid, linolenic acid, omega-3 fatty acids including but not limited to docosahexaenoic acid (DHA) and eicosapentaeonic acid (EPA) and their glycerol-esters. Suitable fat-soluble vitamins include vitamin D (D2, D3 and their derivatives), vitamin E (α, β, γ, δ-tocopherols, or α, β, γ, δ-tocotrienols), vitamin A (retinol, retinal, retinoic acid), vitamin K (K₁, K₂, K₃ and their derivatives) capric/caprylic triglycerides, folic acid, glyceryl linoleate (omega 6 fatty acids vitamin F),

### Cosmeceutical Actives:

aloe vera, beta glucan, bisabolol, camellia thea (green tea) extract, centella asiatica (gotu cola) extract, cetearyl olivate, chlorophyll, citrus sinensis (orange) oil, cocoyl proline, dicapryl ether, disodium lauriminodipropionate tocopheryl phosphates (vitamin E phosphates), glycerin, glyceryl oleate, glycyrrhiza glabra (licorice) root extract, hamamelis virgiana (witch hazel) extract, lactic acid, lecithin, lutein, macadamia integrifolia (macadamia) seed oil, matricaria chamomilla (chamomile) extract, oenothera biennis (evening primrose) oil, olea europaea (olive) leaf extract, rice bran oil, persea gratissima (avocado) oil, polygonum multiflorum extract, pomegranate sterols, resveratrol, rosa eglanteria (rose hip) oil, santalum spicatum (sandalwood) oil, titanium dioxide, tocopheryl phosphates (alpha), vitamin A palmitate, vitis vinifera (grapeseed) oil and zinc oxide,

### Others:

alendronate, traconazole, oxybutynin, phentermine, zegerid, phentermine, zonisamide, metoclopramide, terazosin, acetominophen (paracetamol), gabapentin, baclofen, benzonatate, mometasone furoate, sumatriptan succinate, polyethylene glycol, warfarin, NovoLog, Nitroglycerin, levodopa, cabergoline, clindesse, potassium chloride, PhostLo, medizine hydrochloride, isosorbide monotartrate, lepidopa, nifedical, triamcinolone, lutera, menopur, topiramate, dorzolamide, zopiclone, hydroxychloroquine, nicotine and caffeine.

Particularly preferred biologically active compounds include insulin, alprazolam, lorazepam, donepazil, risperidone, lidocaine, diclofenac, ketoralac, nicotine, and combinations thereof.

It is to be understood that pharmaceutically, nutraceutically or cosmeceutically acceptable derivatives of biologically active compounds are included within the scope of the present invention.

The term “pharmaceutically, nutraceutically or cosmeceutically acceptable derivatives” includes, but is not limited to, pharmaceutically, nutraceutically or cosmeceutically acceptable salts, esters, salts of such esters, ethers, or any other derivative including prodrugs and metabolites, which upon administration to a subject (e.g. patient, human or animal) in need is capable of providing, directly or indirectly, a biologically active compound as otherwise described herein.

As used herein, the term “pharmaceutically, nutraceutically or cosmeceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically, nutraceutically or cosmeceutically acceptable salts are well known in the art. For example, S. M. Berge, et al. describe pharmaceutically, nutraceutically or cosmeceutically acceptable salts in detail in J. Pharmaceutical Sciences, 66:1-19, 1977. Examples of pharmaceutically, nutraceutically or cosmeceutically acceptable nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid, or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hernisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate, and aryl sulfonate.

The term “pharmaceutically, nutraceutically or cosmeceutically acceptable ester” refers to esters which are hydrolysed in vivo and include those that break down readily in the human body to leave the parent compound or a salt thereof. Suitable ester groups include, for example, those derived from pharmaceutically, nutraceutically or cosmeceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include formates, acetates, propionates, butyrates, acrylates and ethylsuccinates.

The term “pharmaceutically, nutraceutically or cosmeceutically acceptable prodrugs” as used herein refers to those prodrugs of the biologically active compounds which are, within the scope of sound medical judgment, suitable for use in contact with the tissues of a subject with undue toxicity, irritation, allergic response, and the like, commensurate with a reasonable benefit/risk ratio, and effective for their intended use, as well as the zwitterionic forms, where possible, of the compounds of the invention. The term “prodrug” refers to compounds that are rapidly transformed in vivo to yield the parent compound of the above formula, for example by hydrolysis in blood. A thorough discussion is provided in T. Higuchi and V. Stella, Pro-drugs as Novel Delivery Systems, Vol. 14 of the A.C.S. Symposium Series, and in Edward B. Roche, ed., Bioreversible Carriers in Drug Design, American Pharmaceutical Association and Pergamon Press, 1987.

The present invention is further not limited solely to the administration of one biologically active compound: more than one biologically active compounds or other therapeutic compounds may be incorporated into the matrix layer.

The biologically active compound may be present in a therapeutically effective amount, that is, an amount necessary to achieve a desired therapeutic effect. Typically, the biologically active compound will be present in an amount of from about 0.1% w/w up to about 30% w/w, up to about 20% w/w, up to about 10% w/w, of the total concentration of the matrix layer. In one embodiment, the matrix layer will have a biologically active compound concentration between about 3.0% w/w up to about 15.0% w/w of the total concentration of the matrix layer.

### Preparation of the Matrix Patch

The matrix patch of the present invention may be prepared by a variety of techniques.

One technique involves combining the polymer carrier and any inert carrier components such as an anti-tacking agent and/or plasticizer with a suitable solvent (e.g. 50% water, 50% ethanol). This is combined with a dispersion comprising the biologically active compound and the phosphate compound of tocopherol, and is stirred until complete homogenisation is achieved. The composition may then be placed in a suitable mould and dried. In a preferred method, the composition may be dried by heating up to about 90° C., preferably for 0.5 to 24 hours. However, formulating and/or drying may be conducted at a temperature within the range of about 30° C. to about 90° C. It has been found that formulating and/or drying at a temperature of about 75° C. results in better delivery of the biologically active compound.

The ratio of components in the composition, that is the biologically active compound:TP ratios may be between about 5:5 to about 5:0.5, with the most preferred value of about 5:1. The polymer carrier:[biologically active compound and TP] is about 1:1 to about 3:1, with preferred values of about 7:6 to about 7:3.

The composition comprising the phosphate compound of tocopherol and the polymer carrier is suitable for use in a transdermal delivery patch for administration of a biologically active compound. This composition essentially forms the matrix layer in a transdermal delivery patch. The matrix layer may be a solid or semi-solid layer.

The transdermal delivery patch usually would also comprise a backing layer. The backing layer acts as a support or substrate for the matrix layer. When preparing a matrix patch using a mould, the backing layer would be placed in the mould before addition of the matrix layer composition.

Accordingly, the matrix layer essentially has two surfaces: a first surface and a second surface opposite the first surface, where the first surface is in contact with the backing layer and the second surface being adapted to be in diffusional contact with the skin of a subject. The subject may be a human or animal.

The present invention therefore also provides use of a matrix patch for transdermal delivery of a biologically active compound, the matrix patch comprising (i) a backing layer, and (ii) a matrix layer which comprises a phosphate compound of tocopherol, a polymer carrier, and a biologically active compound.

Preferably, the backing layer is occlusive or impermeable to protect the matrix layer from the outer environment. However, a non-occlusive backing layer could also be used, so long as the packaging of the matrix patch is fully occlusive to prevent degradation of the matrix layer. An occlusive backing layer is preferred.

The backing layer may be of any thickness, however in the art, backing layers typically have a thickness of about 0.0005 inches to about 0.01 inches.

The present invention therefore provides a transdermal delivery patch for administration of a biologically active compound comprising (i) a backing layer, and (ii) the matrix layer which comprises a phosphate compound of tocopherol, a polymer carrier, and a biologically active compound.

The matrix patch may further comprise a liner which is a removable protective or impermeable layer, usually but not necessarily rendered “non-stick” so as not to stick to the matrix layer. The liner, which may also be referred to as the release liner, protects the matrix patch during storage. During use, the release liner is to be removed.

The liner may be made from the same material as the backing layer, however it may also be a metal foil, Mylar (registered trademark), polyethylene terephthalate, siliconized polyester, fumed silica in silicone rubber, polytretrafluoroethylene, cellophane, siliconized paper, aluminized paper, polyvinyl chloride film, composite foils or films containing polyester such as polyester terephthalate, polyester or aluminized polyester, polytetrafluoroethylene, polyether block amide copolymers, polyethylene methyl methacrylate block copolymers, polyurethanes, polyvinylidene chloride, nylon, silicone elastomers, rubber-based polyisobutylene, styrene, styrene-butadiene, and styrene-isoprene copolymers, polyethylene, and polypropylene.

The release liner may be of any thickness, however in the art, release liners typically have a thickness of about 0.01 mm to about 2 mm.

The matrix patch may also comprise an adhesive layer. The adhesive layer may be an additional layer to the matrix layer, or may be included on the outer margin of the backing layer where the backing layer extends beyond the edges of the matrix layer. Polymeric adhesives useful for transdermal patches include polyacrylate polymers, rubber-based adhesives and polysiloxane adhesives. These types of materials, as well as others, are described by Van Norstrand (The Handbook of Pressure Sensitive Adhesive Technology Second Edition 1989), which is hereby incorporated by reference. Examples of commercially available adhesives include, but are not limited to, polyacrylate adhesives sold under the trademarks DUROTAK (registered trademark) by National Starch and Chemical Corporation, Bridgewater, N.J., as well as GELVA-MULTIPOLYMER SOLUTION (registered trademark) by Cytek Surface Specialties, Smyrna, Ga.

### Advantages

It has surprisingly been found that biologically active compounds can be effectively administered using a transdermal delivery matrix patch comprising a matrix layer which comprises a phosphate compound of tocopherol and a polymer carrier.

Transdermal delivery options for include, for example, topical creams and gels, and skin patches.

Creams and gels may present difficulties with compliance and dosage control, and may be considered messy or unpleasant by patients.

There are different forms of skin patches, including “reservoir” patches and “matrix” patches. Patches may also be single- or multi-layered. A “reservoir” patch essentially has a liquid or gel compartment containing the drug solution or suspension separated by a membrane and a layer of adhesive. In a “matrix” patch, the drug dispersion is present in a semi-solid or solid layer, which may or may not also comprise the adhesive material.

Reservoir patches overcome some of the dosage difficulties with topical creams and gels, however the delivery may be uneven or inconsistent, and there is some risk of perforation of the reservoir. An additional issue relates to delivery of prescribed drugs which may be addictive and subject to abuse. Gels, creams and reservoir patches provide limited barriers to extraction of the drug substance, whereas incorporation of the drug substance within a matrix layer represents a significant, if not almost impossible barrier to extraction of the drug substance.

Delivery of an active orally or by injection typically results in a delivery profile which is non-linear. Transdermal delivery provides a non-invasive way of potentially achieving sustained steady state delivery.

Without wishing to be bound by theory, the presence of a phosphate compound of tocopherol may reduce any skin irritation caused by the biologically active compound and enhance the skin permeation of the biologically active compound. It has also been found that the components of the matrix layer do not formulate well together without the presence of a phosphate compound of tocopherol.

### EXAMPLES

Various embodiments/aspects of the present invention will now be described with reference to the following non-limiting examples.

### Example 1

### Investigation into the Pharmacodynamics of Insulin Formulated into Matrix Patches

Four matrix patches of the present invention were tested against a positive control gel.

The table below sets out the composition of the matrix layer in each of the four matrix patches. The polyvinylpyrrolidone was found to provide the matrix layer with sufficient “tackiness” to avoid the need to include any inert carrier components. The dry weight of each patch was 60 mg.

| Patch No. | TPM | Polyvinylpyrrolidone | Insulin |
|---|---|---|---|
| 1 | 1.2 mg | 54.8 mg | 4 mg |
| | (2% w/w) | (91.33 w/w %) | (6.67 w/w %) |
| 2 | 0.6 mg | 55.4 mg | 4 mg |
| | (1% w/w) | (92.33 w/w %) | (6.67 w/w %) |
| 3 | 0.6 mg | 56.4 mg | 3 mg |
| | (1% w/w) | (94.0 w/w %) | (5 w/w %) |
| 4 | 1.2 mg | 50.8 mg | 8 mg |
| | (2% w/w) | (84.6 w/w %) | (13.33 w/w %) |

The components of the matrix layer were dissolved in a solvent solution (50% water, 50% ethanol). The resulting solution was then poured into individual casts (containing suitable backing layers) at room temperature and the solvent was allowed to evaporate at 75° C. for 1.5 hours.

300 mg of gel was used as the positive control, which comprised 2.25 mg/ml insulin, 2% TPM (2:1), 30% ethanol, 1% carpobol 934 in water adjusted to pH=4.7.

### Study Design

The study was a cross-over design to test the effect of matrix patches of the present invention compared to the gel. In this design, each animal received four of the five treatments across the course of the study. The animals were male and 10-12 weeks of age. Each treatment group was 11 animals. All animals were >300 g in weight, and had circulating glucose concentrations of >10 mmol/L in the fasted state (mean fasted glucose concentration was 21.37±0.85 mmol/L). The key endpoint of the study was blood glucose levels during a 5-hour insulin tolerance test, conducted as described below.

### Streptozotocin Administration

Diabetes was induced by the administration of a single intraperitoneal injection of streptozotocin (STZ) 50 mg/kg (Sigma Chemicals) dissolved in sodium citrate buffer (0.1 mol/L, pH 4.5) immediately before use. Rats were considered diabetic and included in the study if their blood glucose was greater than 16 mmol/L 24 hours after the STZ injection. In all groups blood glucose measurements were made by obtaining a spot sample from tail tipping. Animals were left for 5 days following STZ administration prior to testing.

### Treatment Application

24 hours before the application of the gel and matrix patches the animals were anaesthetised and ˜30 cm² of fur was shaved from the back, avoiding any damage to the skin that could enhance absorption of the formulations. The gel was applied at a dose of 12 mg/cm² across the shaved area. The matrix patches were adhered to the shaved area and protected with the application of a tegaderm dressing. The insulin tolerance tests were performed 24 hours after removing the fur. Following each treatment, the animals were allowed to recover for 3 days before the next treatment.

### ITT (Insulin Tolerance Test)

Animals were fasted for 2 hours prior to the application of insulin or control formulations. Spot blood samples were taken from the tail at 0, 30, 60, 90, 120, 180, 240 and 300 minutes after the application of the gel and matrix patches. Blood glucose levels were determined at the same time points using glucose sticks (AccuChek, Roche Diagnostics).

### Results

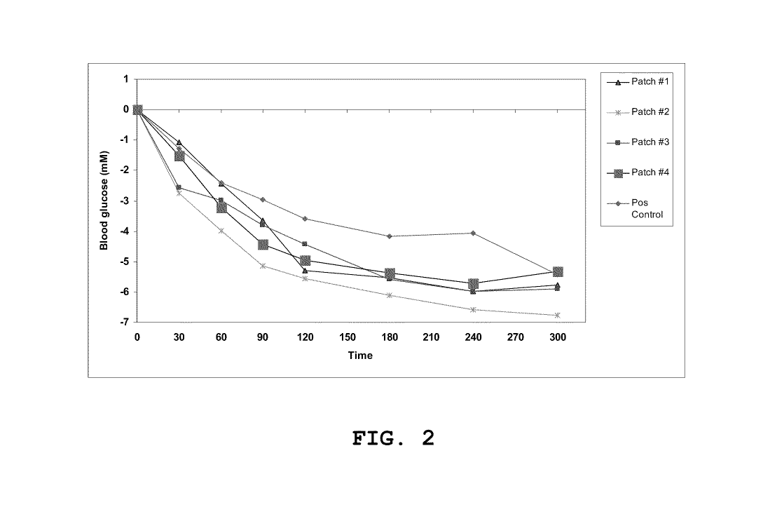

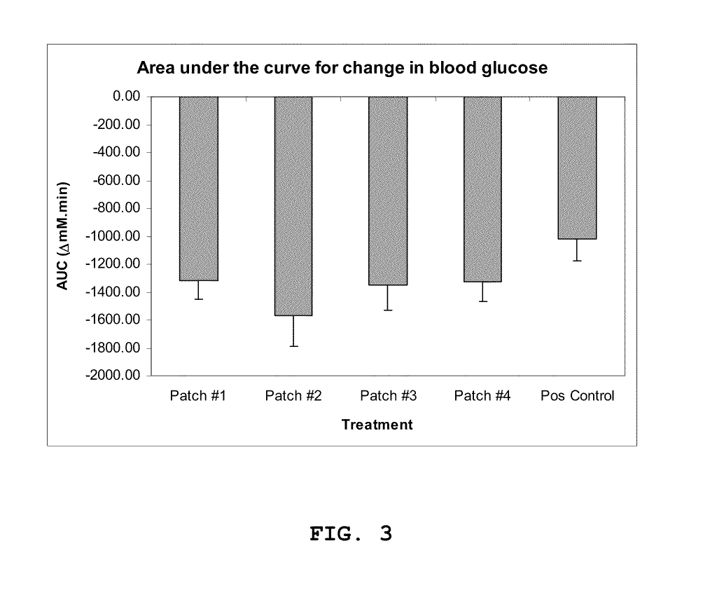

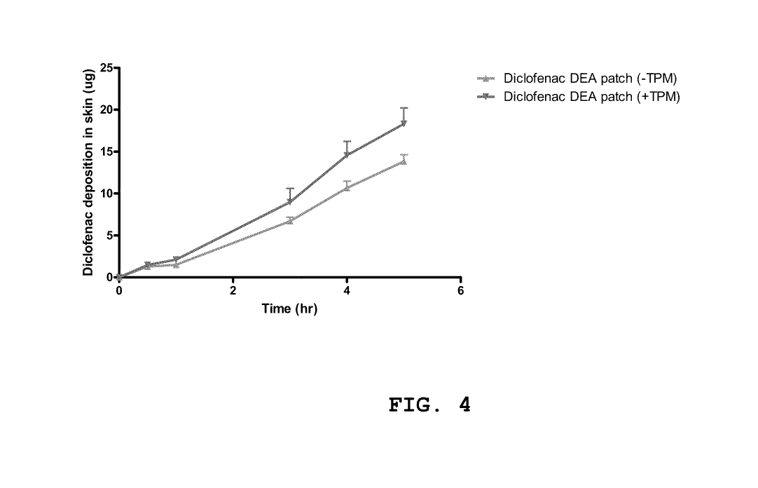

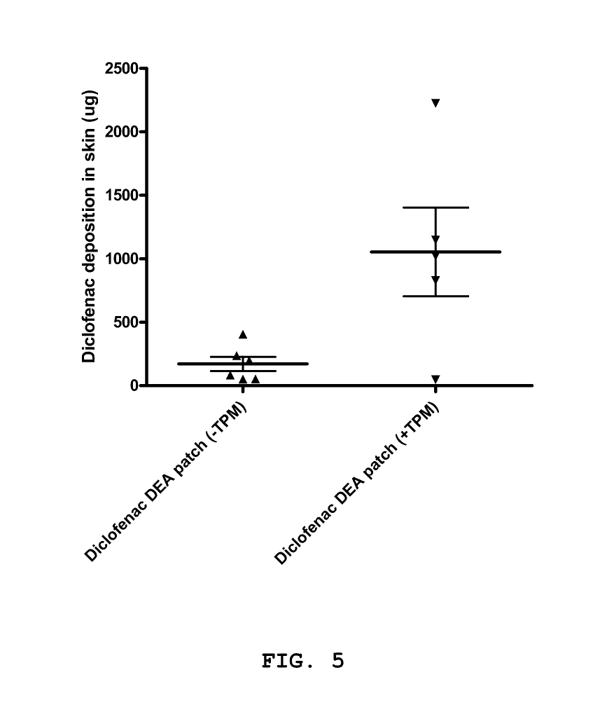

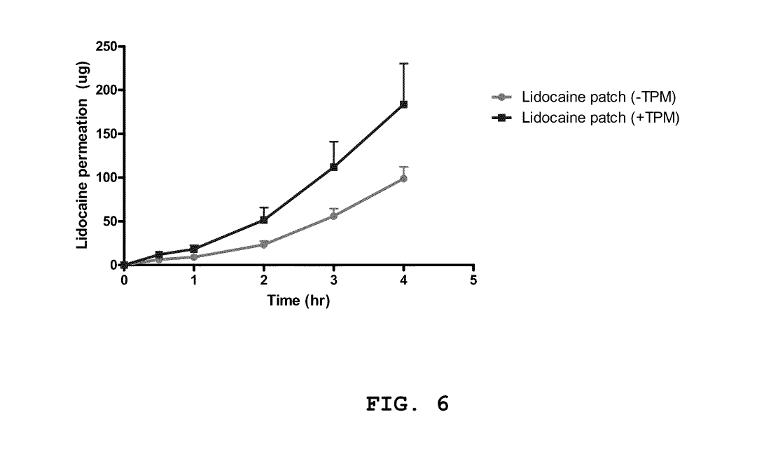

The gel and matrix patches caused significant reductions in blood glucose concentrations in the diabetic rats (see FIGS. 2 and 3). Blood glucose was significantly reduced (p<0.05) from starting values 30 min after application and remained lowered for the duration of the experiment. There was no statistically significant difference in the reduction of blood glucose between the patches and gel tested here, as demonstrated by the area under the curve (see FIG. 3). The matrix patches appear efficacious for the delivery of insulin, however, a matrix patch provides the many advantages described herein over a gel or other method of delivery.

### Example 2

### Lidocaine Matrix Patches

Lidocaine matrix patches were prepared having the following composition:

100 mg lidocaine base
20 mg TPM (8:2)
168 mg Eudragit
100 mg lidocaine base
168 mg Eudragit

The lidocaine matrix patches had a surface area of 120 cm².

### Manufacturing Method

The components listed in the table above were dissolved in 30 ml isopropanol:acetone mixture (1:1) at 45° C. The mixture was then casted over a 3M scotch pack, and dried for 90 minutes at 75° C.

### In-Vitro Testing (Diffusion)

Matrix patches were cut into circular discs (7 cm²) and placed over rat skin. Receptor solution was 12 ml and had an effective surface with the skin equal to about 1.76 cm². After the duration of the experiment, skin (about 7 cm²) was removed, the surface cleaned (excess gel) and extracted with 10 ml solvent.

### Results

| | |
|---|---|
| Diffusion | Dose: 1.46 mg/1.76 cm² |
| Skin extraction | Dose: 5.89 mg/7.06 cm² |

### Example 3

### Diclofenac Matrix Patch

Diclofenac diethylamine matrix patches were prepared having the following composition:

200 mg diclofenac diethylamine
20 mg TPM (8:2)
168 mg Eudragit
200 mg diclofenac diethylamine
168 mg Eudragit

The diclofenac diethylamine matrix patches had a surface area of 120 cm².

### Manufacturing Method

The components listed in the table above were dissolved in 30 ml isopropanol:acetone mixture (1:1) at 45° C. The mixture was then casted over a 3M scotch pack, and dried for 90 minutes at 75° C.

### In-Vitro Testing (Diffusion)

Matrix patches were cut into circular discs (7 cm²) and placed over rat skin. Receptor solution was 12 ml and had an effective surface with the skin equal to about 1.76 cm². After the duration of the experiment, skin (about 7 cm²) was removed, the surface cleaned (excess gel) and extracted with 10 ml solvent.

### Results

| | |
|---|---|
| Diffusion | Dose: 2.92 mg/1.76 cm² |
| Skin extraction | Dose: 11.78 mg/7.06 cm² |

In this specification, except where the context requires otherwise, the words “comprise”, “comprises”, and “comprising” mean “include”, “includes”, and “including” respectively, i.e. when the invention is described or defined as comprising specified features, various embodiments of the same invention may also include additional features.

Although this invention has been described by example and with reference to possible embodiment thereof, it is to be understood that modifications or improvements may be made thereto without departing from the scope of the invention.

1. A composition suitable for use in a transdermal delivery patch for administration of a biologically active compound, the composition comprising a phosphate compound of tocopherol and a polymer carrier.

2. The composition of claim 1, wherein the transdermal delivery patch is a matrix patch.

3. The composition of claim 1, wherein the phosphate compound of tocopherol is selected from the group consisting of mono-(tocopheryl)phosphate, mono-(tocopheryl)phosphate monosodium salt, mono-(tocopheryl)phosphate disodium salt, mono-(tocopheryl)phosphate monopotassium salt, mono-(tocopheryl)phosphate dipotassium salt, di-(tocopheryl)phosphate, di-(tocopheryl)phosphate monosodium salt, di-(tocopheryl)phosphate monopotassium salt, or a mixture thereof.

4. The composition of claim 3, wherein the phosphate compound of tocopherol is present in an amount within the range of about 0.01% w/w to about 10% w/w, about 0.1% w/w to about 5% w/w, about 0.5% w/w to about 2% w/w or to about 3% w/w, of the total concentration of the composition.

5. The composition of claim 4, wherein the phosphate compound of tocopherol is present in an amount of about 1% w/w to about 1.5% w/w of the total concentration of the composition.

6. The composition of claim 1, wherein the polymer carrier comprises natural and synthetic polymers, co-polymers, or terpolymers.

7. The composition of claim 6, wherein the polymer carrier comprises polyvinyl pyrrolidone, polysiloxanes or polymethyl methacrylate.

8. The composition of claim 6, wherein the polymer carrier is present in an amount of from about 20% w/w up to about 90% w/w, from about 30% w/w up to about 80% w/w, from about 55% w/w up to about 65% w/w, of the total weight of the composition.

9. The composition of claim 6, wherein the polymer carrier also comprises an inert carrier component selected from the group consisting of anti-tacking agents, tackifiers, and plasticizers.

10. The composition of claim 9, wherein the inert carrier components is present in an amount of from 0.001% w/w up to about 50% w/w or up to about 40% w/w or up to about 30% w/w, of the total weight of the composition.

11. Use of a phosphate compound of tocopherol and a polymer carrier as a matrix layer in a transdermal delivery patch for administration of a biologically active compound.

12. A transdermal delivery patch for administration of a biologically active compound comprising (i) a backing layer, and (ii) a matrix layer, which comprises a composition of claim 1, and a biologically active compound.

13. The transdermal delivery patch of claim 12, wherein the biologically active compound is selected from the group consisting of pharmaceuticals including drugs, cosmeceuticals, nutraceuticals, and nutritional agents, and pharmaceutically-acceptable salts, prodrugs, or derivatised compounds thereof.

14. The transdermal delivery patch of claim 13, wherein the biologically active compound is insulin.

15. The transdermal delivery patch of claim 12, wherein the biologically active compound is present in an amount of from about 0.1% w/w up to about 30% w/w or up to about 20% w/w or up to about 10% w/w, of the total concentration of the matrix layer.

16. The transdermal delivery patch of claim 12, wherein the biologically active compound is present in an amount of about 4.5% w/w to about 5.5% w/w of the total concentration of the matrix layer.

17. The transdermal delivery patch of claim 12, wherein the backing layer is occlusive.

18. Use of a matrix patch for transdermal delivery of a biologically active compound, the matrix patch comprising (i) a backing layer and (ii) a matrix layer, which comprises a composition of claim 1, and a biologically active compound.

19. A method for preparing a transdermal delivery patch for administration of a biologically active compound comprising the steps of:
(i) combining a polymer carrier and optional inert carrier components with a suitable solvent;
(ii) combining (i) with a dispersion comprising a phosphate compound of tocopherol and a biologically active compound;
(iii) stirring (ii) until complete homogenisation is achieved;
(iv) placing (iii) in a mould comprising a suitable backing layer; and
(v) drying the compositions in the mould by heating them up to about 90° C. for about 0.5 to about 24 hours.

20. The method of claim 19, wherein drying is conducted at a temperature of 75° C.
